(19) United States
(12) Patent Application Publication
(10) Pub. No.: US 2023/0027104 A1
(43) Pub. Date: Jan. 26, 2023

(54) NUTRITIONAL POWDER COMPRISING OLIGOPEPTIDES AND APPLICATIONS THEREOF

(71) Applicant: Suzhou Jianyuan Biotechnology Co., Ltd., Suzhou (CN)

(72) Inventors: Yuhong ZHENG, Suzhou (CN); Qinghua LIU, Suzhou (CN); Jia DU, Suzhou (CN); Cheng ZHONG, Suzhou (CN); Naomi SUDO, Suzhou (CN)

(21) Appl. No.: 17/412,232

(22) Filed: Aug. 25, 2021

(30) Foreign Application Priority Data
Jul. 13, 2021 (CN) 202110788989.7

(51) Int. Cl. A61K 36/899; A23L 33/18; A23L 33/105; A23L 33/175; A23L 33/15; A23L 33/16; A23L 27/30; A61K 36/48; A61K 35/60; A61K 36/258; A61K 36/52; A61K 36/72; A61K 36/185; A61K 36/73; A61K 36/28; A61K 31/198; A61K 31/401; A61K 31/51; A61K 31/525; A61K 31/455; A61K 31/4415; A61K 31/714; A61K 31/519; A61K 31/4188; A61K 33/30; A61K 33/06; A61P 39/00

(52) U.S. Cl. CPC A61K 36/899; A23L 33/18; A23L 33/105; A23L 33/175; A23L 33/15; A23L 33/16; A23L 27/33; A61K 36/48; A61K 35/60; A61K 36/258; A61K 36/52; A61K 36/72; A61K 36/185; A61K 36/73; A61K 36/28; A61K 31/198; A61K 31/401; A61K 31/51; A61K 31/525; A61K 31/455; A61K 31/4415; A61K 31/714; A61K 31/519; A61K 31/4188; A61K 33/30; A61K 33/06; A61P 39/00; A23V 2002/00

(57) ABSTRACT

A nutritional powder containing oligopeptide includes the following components by weight: oligopeptide powder 55-80%, fruit powder 5-30%, amino acid powder 0.5-5%, multivitamin powder 1.25-5%, mineral powder 0.25-3%, and compound sweetener powder 5-10%.

### CROSS-REFERENCE TO RELAYED APPLICATIONS

Pursuant to 35 U.S.C. § 119 and the Paris Convention Treaty, this application claims foreign priority to Chinese Patent Application No. 202110788989.7 filed Jul. 13, 2021, the contents of which, including any intervening amendments thereto, are incorporated herein by reference. Inquiries from the public to applicants or assignees concerning this document or the related applications should be directed to: Matthias Scholl P. C., Attn.: Dr. Matthias Scholl Esq., 245 First Street, 18th Floor, Cambridge, Mass. 02142.

### BACKGROUND

The disclosure relates to a nutritional powder comprising oligopeptide and applications thereof.

Excessive drinking and drunkenness usually lead to hangover symptoms. Hangover symptoms are manifestations of ethanol and acetaldehyde poisoning of human body, which is caused by alcohol-induced oxidative stress and inflammatory response.

The metabolic pathways of ethanol degradation, oxidative stress, inflammatory response and hangover symptom are shown in Metabolic Pathway 1 in FIG. 1. As shown in Metabolic Pathway 2 in FIG. 1, a large amount of oxidized free radicals and acetaldehyde are produced during the metabolism of ethanol and acetaldehyde. Acetaldehyde is a recognized carcinogen, and tends to react with ethanol's metabolic intermediates to produce an acetaldehyde-protein adduct, causing alcohol inflammatory reactions, thereby aggravating hangover symptoms and causing great toxicity and physiological damage to human body.

To reduce the harm of alcoholic intoxication, comprehensive measures such as detoxification, taking antialcoholismic drugs and physical recovery are essential. However, the existing health care products for hangover cure or antialcoholism involve in several antioxidant components and vitamins, cysteine or a certain low molecular weight peptide. These products aim to dispel the initial effects of alcohol and alleviate a hangover, but ignore the comprehensive measures for physical recovery and health care of secondary symptoms based on the pathogenesis of alcohol hangover.

In addition, tablets or capsules are used to cure hangover but cannot be quickly absorbed by human body, making it difficult to achieve the effect of rapid alleviation and recovery from severe hangover. Some antialcoholismic drugs are liquid; however, the active ingredients thereof are unstable in a liquid state.

### SUMMARY

One objective of the disclosure is to provide a nutritional powder comprising oligopeptides, to solve the problem of single component, single function, and insignificant effects of hangover cure, cell repair and rehabilitation of antialcoholismic drugs in the relevant art. Based on the latest biotechnology research results of biometabolic pathway of ethanol, drunkenness pathology and hangover recovery, a comprehensive approach with supplements of oligopeptides and micronutrients and synergistic methods can be developed to strengthen the activity of alcohol dehydrogenase and acetaldehyde dehydrogenase, to reduce oxidative stress and to slow down the inflammatory response, thereby reducing the harm of alcohol to the human body.

Another objective of the disclosure is to provide the applications of the nutritional powder with oligopeptides in preparing antialcoholismic drugs or health care products.

The oligopeptide nutritional powder provided in the disclosure comprises the following components by weight: oligopeptide powder 55-80%, fruit powder 5-30%, amino acid powder 0.5-5%, multivitamin powder 1.25-5%, mineral powder 0.25-3%, and compound sweetener powder 5-10%.

In a class of this embodiment, the oligopeptide powder comprises the following components by weight: soybean oligopeptide powder 40-55%, shad egg oligopeptide powder 10-20%, corn oligopeptide powder 10-20%, ginseng oligopeptide powder 5-10%, rice oligopeptide powder 5-10%, and walnut oligopeptide powder 5-10%.

In a class of this embodiment, the fruit powder comprises the following components by weight: Hovenia dulcis powder 20-40%, sea buckthorn powder 20-30%, pear powder 20-30%, milk thistle powder 5-10%, and Aronia melanocarpa powder 5-20%.

In a class of this embodiment, the amino acid powder comprises the following components by weight: cysteine powder 40-60%, proline powder 20-40%, and threonine powder 10-30%.

In a class of this embodiment, the multivitamin powder is a complex B vitamin and comprises the following components by weight: vitamin B1 20-40%, vitamin B2 20-40%, vitamin B3 10-20%, vitamin B6 15-25%, vitamin B9 1.5-5%, vitamin B12 0.5-2%, and vitamin B7 0.2-0.8%.

In a class of this embodiment, the mineral powder comprises the following components by weight: food-grade zinc gluconate powder 30-70%, and food-grade magnesium sulfate powder 30-70%.

In a class of this embodiment, the compound sweetener powder is Momordica grosvenori powder, arabinose, or a mixture thereof.

The disclosure further provides a method for hangover cure comprising administering to a drunken person in need thereof the composition of matter.

The disclosure proposes a scientific and comprehensive complementary synergistic formula comprising a variety of oligopeptides and micronutrient components using the comprehensive evaluation and function optimization technology of micronutrient combination, to alleviate the discomfort, poisoning and physiological harms to the body caused by drunkenness from three paths of fast elimination, prevention of brain damage, detoxification or anti-inflammation and damage recovery. Specifically, as shown in FIG. 2, the activity of related enzymes is activated by zinc, niacin, cysteine (Cys) and corn oligopeptide (COP), etc. to accelerate the metabolism of ethanol and convert acetaldehyde into non-toxic acetic acid, reducing the content of ethanol; or ethanol or acetaldehyde is neutralized or combined by active substances such as Cys, to reduce their free components in the body and form non-toxic MTCA to eliminate ethanol from the body; then ethanol is prevented from entering the brain by Cys and other micronutrients, to reduce the effect of drunkenness; Finally, under the complementary and synergistic effect of a variety of antioxidant combinations (various natural antioxidant components, such as shad egg peptide, glutathione, ginseng peptide, corn peptide and other functional oligopeptides, a variety of fruit active substances, Cys and other amino acids, B vitamins), ethanol or acetaldehyde is degraded by the mitochondrial ethanol oxidation system (MEOS), to reduce the toxicity of ethanol and acetaldehyde and the free radicals generated in the metabolic process, and alleviate the damage to cells and tissues caused by oxidative stress and inflammatory response; at the same time, through the micronutrient supplement (including a variety of soybean oligopeptides, shad egg peptides, rice oligopeptides, fruit powder and vitamins, etc.), the damage recovery and physical rehabilitation are accelerated. The formula of the oligopeptide nutritional powder mainly includes natural plant extracts in the disclosure. A variety of oligopeptides (low molecular weight peptides) used such as glutathione (GSH), ginseng oligopeptide (GOP), rice oligopeptide (ROP), COP, etc. are oligopeptides, extracted from soybeans, shad egg peptides, ginsengs, rice and corns, etc. and compatible with natural antioxidant substances (fruit powders) and vitamins, Cys and other trace elements. These components have complementary and synergistic effects, which can enhance immune functions and have the anti-oxidation, anti-stress, anti-inflammatory, blood pressure lowering, cholesterol-lowering, anti-fatigue and other special comprehensive effects. In addition, the powder formulated with the above micronutrients has high solubility, can be absorbed by the small intestine in a short time to directly enter the blood circulation and rapidly take their biological effects.

In summary, through the comprehensive complementary and synergistic effects of a variety of oligopeptides with fruit powders and other natural antioxidants, amino acids, vitamins and minerals, etc., the excretion and metabolism of ethanol are promoted to “detoxify” and reduce the content of ethanol and acetaldehyde; the body achieves “detoxification” through enhancing the body's anti-oxidation, anti-stress and anti-inflammatory effects, to reduce and eliminate the physiological hazards caused by drunkenness; finally through the micronutrient supplementation, the damage to organs and tissues caused by drunkenness is “repaired”, to quickly recover body health condition. The oligopeptide nutritional powder is a powder with instant solubility property, which can be quickly dissolved in water to make instant drink. The instant drink has the characteristics of fruity flavor and deliciousness. It can be quickly absorbed directly in the small intestine without need of digestion after drinking, to achieve the function of protecting and repairing organs and tissues such as the liver in a short time.

The following advantages are associated with the composition of matter of the disclosure. The oligopeptide nutritional powder of the disclosure comprises a variety of oligopeptide powders and micronutrient components from natural food materials, which is a comprehensive formula integrating the functions of antioxidation, detoxification, liver protection, brain strengthening, tissue repair, anti-aging, etc. to achieve elimination of toxicant, detoxification and health restoration from three aspects. Among them, soybean oligopeptides are rich in a variety of essential amino acids, which have strong antioxidant functions, especially protecting and repairing the damage caused by acetaldehyde oxidation. Shad egg peptides are rich in the main amino acids of antioxidant active polypeptides, including tryptophane, cysteine, histidine, lysine, and methionine, which have as strong function of scavenging free radicals. Ginseng oligopeptides can enhance immunity and promote the repair of damaged tissues. Corn oligopeptides have a strong hepatoprotective effect and can enhance the ability of ethanol degradation. Walnut oligopeptides have strong antioxidant, anti-inflammatory and brain-invigorating functions, playing an important role in reducing the damage of ethanol to the brain. Rice oligopeptides facilitate to improve the immunity and antioxidant ability and have an important complementary and synergistic effect on liver protection and physical rehabilitation. Among the fruit powders, Hovenia dulcis powder is rich in dihydromyricetin (DHM) which has a strong anti-alcoholic ability. Pear and sea buckthorn powder contain a variety of biological enzymes and vitamin C that have oxidative and liver-protecting functions. A number of vitamins and minerals, especially B vitamins, have strong antioxidant capacity, or involve in the metabolism and repair of energy, nerves, digestion and immune functions as coenzymes. Zinc ion has a strong enzymatic function, especially can increase the activity of alcohol dehydrogenase and acetaldehyde dehydrogenase. Magnesium ion is involved in the metabolic functions of nerve, muscle and heart, etc., to facilitate physical restoration. Milk thistle contains silymarin, an important protective agent for glutathione, can facilitate to improve the anti-alcoholic function, antioxidant and resistance to disease. The above natural components combine complementarily and synergize, to reduce alcohol poisoning, protect internal organs and promote physical rehabilitation.

### BRIEF DESCRIPTION OF THE DRAWINGS

FIG. 1 is a metabolic pathway diagram of ethanol degradation, oxidative stress, inflammatory response and hangover symptoms in the relevant art;

FIG. 2 is a diagram of three pathways of fast elimination, detoxification and damage recovery from hangovers;

FIG. 3 is a comparison chart of a 12-hour alleviation effect of a nutritional powder comprising oligopeptide of the disclosure after excessive drinking of liquors;

FIG. 4 is a comparison chart of a 12-hour alleviation effect of a nutritional powder comprising oligopeptide of the disclosure after excessive drinking of red wine;

FIG. 5 is a comparison chart of a 36-hour alleviation effect of a nutritional powder comprising oligopeptide of the disclosure after excessive drinking of liquors; and

FIG. 6 is a comparison chart of a 36-hour alleviation effect of a nutritional powder comprising oligopeptide of the disclosure after excessive drinking of red wine.

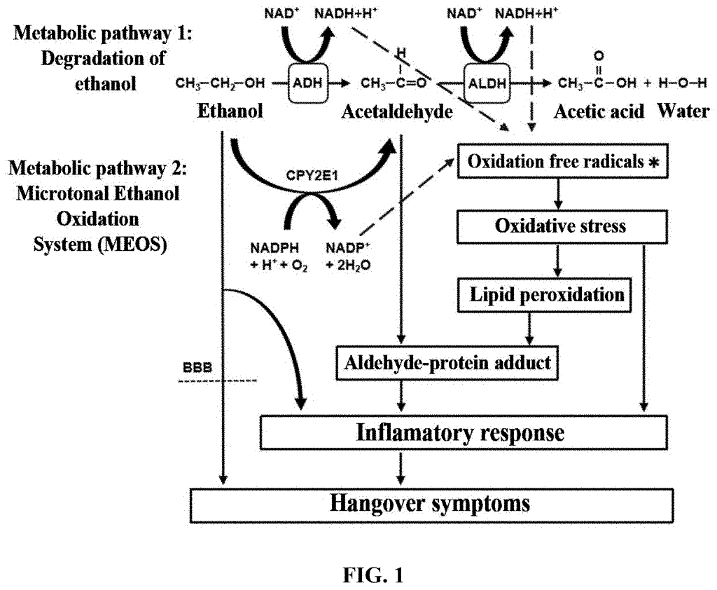

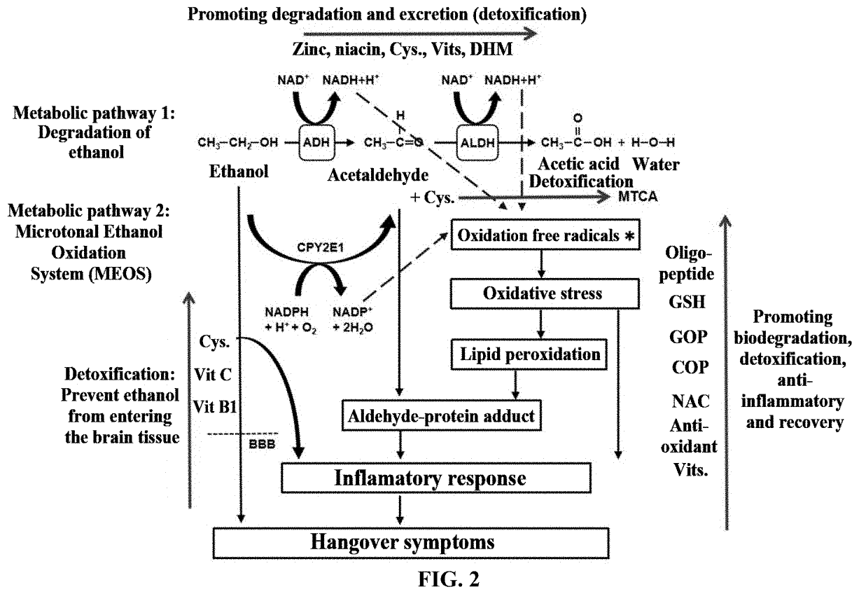

### DETAILED DESCRIPTION

To further illustrate, embodiments detailing a nutritional powder comprising oligopeptide are described below. It should be noted that the following embodiments are intended to describe and not to limit the disclosure.

All materials used in the disclosure are food or pharmaceutical raw materials that are legally permitted and conform to the requirements of pharmacopoeia and other standards, and they are commercially available unless otherwise specified.

### Example 1 Preparation of Oligopeptide Nutritional Powder

The oligopeptide micro-nutritive health care foods for hangover and rehabilitation were prepared according to the following formula by weight: oligopeptide powder 55%, fruit powder 30%, amino acid powder 5%, multivitamin powder 4.75%, mineral powder 0.25% and compound sweetener powder 5%. Among them, the oligopeptide powder was prepared according to the following components (by weight): soybean oligopeptide powder 40%, shad egg oligopeptide powder 20%, corn oligopeptide powder 20%, ginseng oligopeptide powder 10%, and rice oligopeptide powder 5%, walnut oligopeptide powder 5%. The fruit powder was prepared according to the following components (by weight): Hovenia dulcis powder 20%, sea buckthorn powder 30-%, pear powder 30%, milk thistle powder 5%, Aronia melanocarpa powder 15%. The amino acid powder was prepared according to the following components (by weight): cysteine powder 40%, proline 40%, threonine 20%. The complex B vitamins (by weight) contained B1 20%, B2 40%, B3 (niacin) 20%, B6 17.8%, B9 (folate) 1.5%, B12 0.5%, B7 0.2%. The minerals (by weight) contained food grade zinc gluconate powder 30%, food grade magnesium sulfate powder 70%. The compound sweetener powder (by weight) contained Momordica grosvenori powder 100%. The above nutritional powders were placed into an ultrafine pulverizer by a certain ratio, mixed to make ultrafine powder, then made into small bags of 10 g oligopeptide nutritional powder by a full-automatic back-seal powder packaging machine.

### Example 2 Preparation of Oligopeptide Nutritional Powder

The oligopeptide micro-nutritive health care foods for hangover and rehabilitation were prepared according to the following formula by weight: oligopeptide powder 80%, fruit powder 5%, amino acid powder 0.5%, multivitamin powder 1.5%, mineral powder 3% and compound sweetener powder 10%. Among them, the oligopeptide powder was prepared according to the following components (by weight): soybean oligopeptide powder 55%, shad egg oligopeptide powder 10%, corn oligopeptide powder 10%, ginseng oligopeptide powder 5%, and rice oligopeptide powder 10%, walnut oligopeptide powder 10%. The fruit powder was prepared according to the following components (by weight): Hovenia dulcis powder 40%, sea buckthorn powder 20%, pear powder 20%, milk thistle powder 10%, Aronia melanocarpa powder 10%. The amino acid powder was prepared according to the following components (by weight): cysteine powder 60%, proline 20%, threonine 20%. The complex B vitamins (by weight) contained B1 40%, B2 20%, B3 (niacin) 10%, B6 22.2%, B9 (folate) 5%, B12 2%, B7 0.8%. The minerals (by weight) contained food grade zinc gluconate powder 70%, food grade magnesium sulfate powder 30%. The compound sweetener powder (by weight) contained arabinose 100%. The above nutritional powders were placed into an ultrafine pulverizer by a certain ratio, mixed to make ultrafine powder, then made into small bags of 8 g oligopeptide nutritional powder by a full-automatic back-seal powder packaging machine.

### Example 3 Preparation of Oligopeptide Nutritional Powder

The oligopeptide micro-nutritive health care foods were prepared according to the following formula by weight: oligopeptide powder 65%, fruit powder 25%, amino acid powder 2.0%, multivitamin powder 2.2%, mineral powder 0.8% and compound sweetener powder 5%. Among them, the oligopeptide powder was prepared according to the following components (by weight): soybean oligopeptide powder 42%, shad egg oligopeptide powder 18%, corn oligopeptide powder 20%, ginseng oligopeptide powder 6%, and rice oligopeptide powder 6%, walnut oligopeptide powder 8%. The fruit powder was prepared according to the following components (by weight): Hovenia dulcis powder 32%, sea buckthorn powder 22%, pear powder 26%, milk thistle powder 8%, Aronia melanocarpa powder 12%. The amino acid powder was prepared according to the following components (by weight): cysteine powder 55%, proline 35%, threonine 10%. The complex B vitamins (by weight) contained B1 30%, B2 25%, B3 (niacin) 20%, B6 20%, B9 (folate) 3.5%, B12 1%, B7 0.5%. The minerals (by weight) contained food grade zinc gluconate powder 80%, food grade magnesium sulfate powder 20%. The compound sweetener powder (by weight) contained Momordica grosvenori powder 80%, arabinose 20%. The above nutritional powders were placed into an ultrafine pulverizer by a certain ratio, mixed to make ultrafine powder, then made into small bags of 5 g oligopeptide nutritional powder by a full-automatic back-seal powder packaging machine.

### Example 4 Dissolution Rate of Oligopeptide Nutritional Powder

10 grams of the oligopeptide nutritional powder prepared in Example 3 were put into 6 cups, and then 150 mL of plain boiled water at different temperatures (20° C., 30° C., 35° C., 40° C., 50° C. and 60° C.) were added to each cup, to observe the time it took for the nutritional powder to change from a turbid liquid to a transparent liquid, namely, the time to dissolve the nutritional powder completely (dissolution time). Details were shown in Table 1.

TABLE 1
Dissolution time of oligopeptide nutritional
powder at different temperatures

| Group | 20° C. | 30° C. | 35° C. | 40° C. | 50° C. | 60° C. |
|---|---|---|---|---|---|---|
| Dissolution | 400 | 180 | 150 | 120 | 90 | 60 |
| time (seconds) | | | | | | |

As shown from Table 1, in 20° C. plain boiled water, it took nearly 7 minutes to dissolve oligopeptide nutritional powder completely; in 30° C. plain boiled water, it took 3 minutes to dissolve oligopeptide nutritional powder completely; in 40° C. plain boiled water, it took 2 minutes to dissolve oligopeptide nutritional powder completely; in 50° C. plain boiled water, it took 90 seconds to dissolve oligopeptide nutritional powder completely; and in 60° C. plain boiled water, it took 60 seconds to dissolve oligopeptide nutritional powder completely. By comprehensive analysis of the dissolution time and palatability of the oligopeptide nutritional powder, 30-40° C. were the appropriate dissolution temperatures.

### Example 5 Hangover Cure and Rehabilitation Effect of Oligopeptide Nutritional Powder for Excessive Drinking of Liquors

Thirty-six healthy middle-aged men and women were chosen and divided into four groups, namely, the control group, the cysteine group, the 5 g oligopeptide nutrition group (prepared in Example 3) and the 10 g oligopeptide nutrition group (prepared in Example 3). Ten to twenty minutes before dinner (at time 18:00), 150 mL of plain boiled water at a temperature of 35° C. was prepared for each person in advance. In the control group, the subjects only drank plain boiled water; in the cysteine group, the subjects took 1 cysteine tablet (each comprising 1200 mg L-cysteine, produced by U.S. Now Food) when drinking water; in the 5 g oligopeptide nutritional group, the subjects put 5 g of the oligopeptide nutritional powder into a cup, poured 150 mL of plain boiled water, stirred to dissolve and drink; in the 10 g oligopeptide nutritional group, the subjects put 10 g of the oligopeptide nutritional powder into a cup, poured 150 mL of plain boiled water, stirred to dissolve and drink.

Dinners started at 18:00 and ended at 20:00. Each of 36 subjects drank 200 mL of liquor (Kweichow Moutai Liquor 2018, 53% vol. 106 PROOF) within two hours. The subjects' dinners were mainly Chinese stir-fried dishes, including fish, meat and vegetables, with rice as the staple food, and the dinners lasted two hours. In the next morning at 8:00 (12 hours after drinking) and in the third morning at 8:00 (36 hours after drinking), the subjects filled in the forms, indicating personal feelings. The personal feelings included 5 levels, namely, level 0 indicated no discomfort, level 1 indicated slight tiredness, level 2 indicated feeling of dizziness, level 3 indicated headache, level 4 indicated nausea or vomiting, requiring stay in bed. All subjects' forms were collected, and the subjects' conditions were recorded and analyzed by classification.

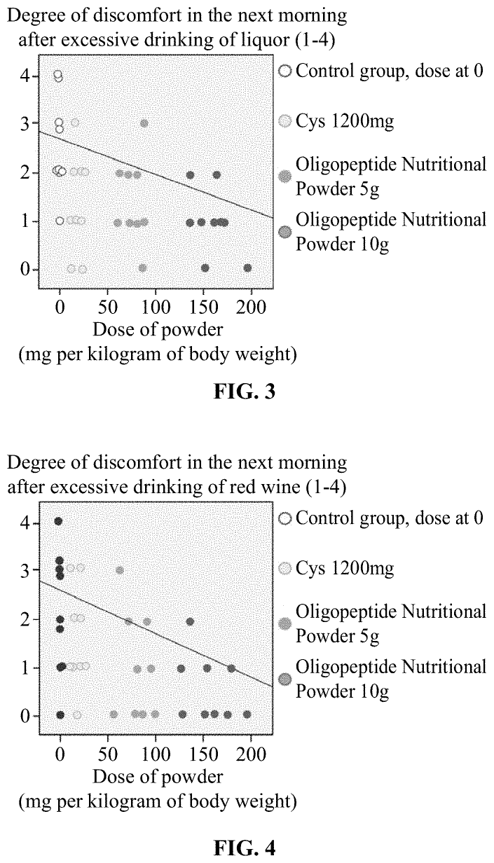

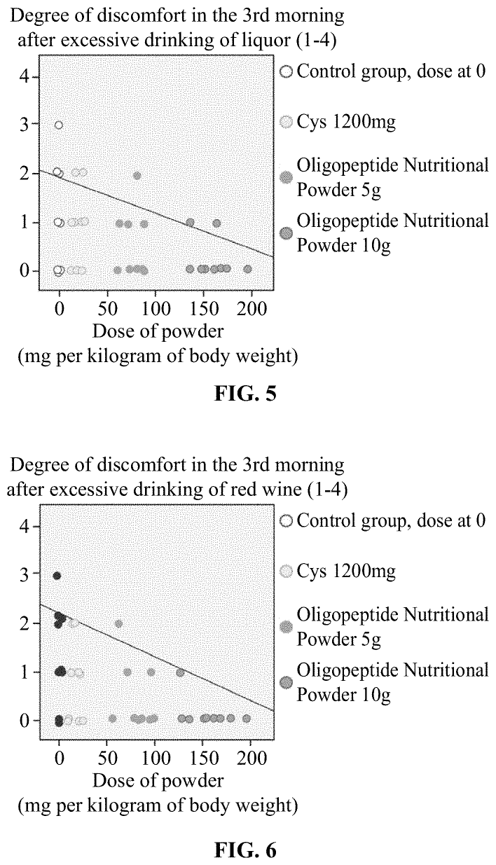

As shown in FIG. 3, at 8:00 the next morning after drinking 200 mL of liquor (12 hours after drinking), 88.9% of the subjects had obvious discomfort (>2 degree of discomfort) in the next morning among the populations in the control group; 44.4% of the subjects had obvious discomfort in the next morning among the populations who took cysteine tablets; 44.4% of the subjects had obvious discomfort in the next morning among the populations who took 5 g of the oligopeptide nutritional powder; and only 22.2% subjects had obvious discomfort in the next morning among the populations who took 10 g of the oligopeptide nutritional powder, indicating that subjects who took 10 g of the oligopeptide nutritional powder had a significant hangover effect. As shown in FIG. 5, at 8:00 the third morning (36 hours after drinking), 33.3% of the subjects still had obvious discomfort among the populations in the control group; 22.2% of the subjects had obvious discomfort among the populations who took cysteine tablets; only 11.1% of the subjects had obvious discomfort among the populations who took 5 g of the oligopeptide nutritional powder; and all subjects recovered to normal among the populations who took 10 g of the oligopeptide nutritional powder, indicating that the oligopeptide nutritional powder of the disclosure has a significant rehabilitation effect on overdose of liquor.

### Example 6 Hangover Cure and Rehabilitation Effect of Oligopeptide Nutritional Powder for Excessive Drinking of Red Wine

Thirty-six healthy middle-aged men and women were chosen and divided into four groups, namely, the control group, the cysteine group, the 5 g oligopeptide nutrition group (prepared in Example 3) and the 10 g oligopeptide nutrition group (prepared in Example 3), and they experienced the hangover cure and rehabilitation effect of the oligopeptide nutritional powder for excessive drinking of red wine. Ten to twenty minutes before dinner, 150 mL of plain boiled water at a temperature of 35° C. was prepared for each person. In the control group, the subjects only drank plain boiled water; in the cysteine group, the subjects took 1 cysteine tablet (each comprising 1200 mg L-cysteine, produced by U.S. Now Food) when drinking water; in the 5 g oligopeptide nutritional group, the subjects put 5 g of the oligopeptide nutritional powder into a cup, poured 150 mL of plain boiled water, stirred to dissolve and drink; in the 10 g oligopeptide nutritional group, the subjects put 10 g of the oligopeptide nutritional powder into a cup, poured 150 mL of plain boiled water, stirred to dissolve and drink.

Dinners started at 18:00 and ended at 20:00. Each of 36 subjects drank 400 mL of red wine (France Saint Arnaud Cabernet Sauvignon 2015, 13.5% vol. Cabernet Sauvignon). The subjects' dinners were mainly Chinese stir-fried dishes, including fish, meat and vegetables, with rice as the staple food, and the dinners lasted two hours. In the next morning at 8:00 (12 hours after drinking) and in the third morning at 8:00 (36 hours after drinking), the subjects filled in the forms, indicating personal feelings. The personal feelings included 5 levels, namely, level 0 indicated no discomfort, level 1 indicated slight tiredness, level 2 indicated feeling of dizziness, level 3 indicated headache, level 4 indicated nausea or vomiting, requiring stay in bed. All subjects' forms were collected, and the subjects' conditions were recorded and analyzed by classification.

As shown in FIG. 4, at 8:00 the next morning after drinking 400 mL of red wine (12 hours after drinking), 66.7% of the subjects had obvious discomfort (>2 degree of discomfort) in the next morning among the populations in the control group; 44.4% of the subjects had obvious discomfort in the next morning among the populations who took cysteine tablets; 33.3% of the subjects had obvious discomfort in the next morning among the populations who took 5 g of the oligopeptide nutritional powder; and only 11.1% subjects had obvious discomfort in the next morning among the populations who took 10 g of the oligopeptide nutritional powder, indicating that subjects who took 10 g of the oligopeptide nutritional powder had a significant hangover effect for red wine. As shown in FIG. 6, at 8:00 the third morning (36 hours after drinking), 44.4% of the subjects still had obvious discomfort among the populations in the control group; 22.2% of the subjects had obvious discomfort among the populations who took cysteine tablets; only 11% of the subjects had obvious discomfort among the populations who took 5 g of the oligopeptide nutritional powder; and all subjects recovered to normal among the populations who took 10 g of the oligopeptide nutritional powder, indicating that the oligopeptide nutritional powder of the disclosure has a significant hangover cure and rehabilitation effect for red wine.

According to the above results, although taking cysteine tablets had a certain hangover effect, there was significant difference in the hangover and physical rehabilitation for excessive drinking of liquor and red wine from taking oligopeptide nutritional powder. As shown in Table 2, the beneficial effects of taking oligopeptide nutritional powder were as follows: within 12 hours, only 16.7% of the subjects who took 10 g of the oligopeptide nutritional powder had obvious discomfort, and in contrast, 44.4% of subjects who took cysteine tablets had no obvious discomfort; and within 36 hours, all subjects who took 10 g of the oligopeptide nutritional powder recover to normal health condition, while 22.2% of subjects still had discomfort in the cysteine group, demonstrating that the active ingredients of the oligopeptide nutritional powder comprehensively reduced the damage of ethanol to the brain tissues (sobering), reduced the damage of free radicals such as acetaldehyde to the body (hangover), accelerated the nutritional repair of cells, protected the liver and other tissues, and helped to recover to normal health conditions (rehabilitation), with a very significant effect.

TABLE 2
Summary of hangover cure and rehabilitation effects of oligopeptide
nutritional powder on excessive drinking of liquor and red wine

| | | | Oligopeptide | Oligopeptide |
|---|---|---|---|---|
| Group | Control group | Cysteine | nutritional | nutritional |
| Percentage of | (no health | group | powder | powder |
| discomfort | care products) | (1200 mg) | (5 g) | (10 g) |
| Proportion | 77.8%^{a} | 44.4%^{b} | 38.9%^{c} | 16.7%^{d} |
| of > degree | | | | |
| 2 discomfort 12 | | | | |
| hours after | | | | |
| drinking wine | | | | |
| Proportion | 38.9%^{a} | 22.2%^{a} | 11%^{b} | 0%^{c} |
| of > degree | | | | |
| 2 discomfort 36 | | | | |
| hours after | | | | |
| drinking wine | | | | |

### Example 7 Effect of Oligopeptide Nutritional Powder on Blood Alcohol Content after Drinking Wines

Thirty healthy middle-aged men and women were chosen and divided into three groups, namely, the control group, the cysteine group and the 10 g oligopeptide nutrition group. After drinking liquor and red wine, the effects of the oligopeptide nutritional powder on the blood alcohol contents were compared. Ten to twenty minutes before dinner, 150 mL of plain boiled water at a temperature of 35° C. was prepared for each person. In the control group, the subjects only drank plain boiled water; in the cysteine group, the subjects took 1 cysteine tablet (each comprising 1200 mg L-cysteine, produced by U.S. Now Food) when drinking water; in the 10 g oligopeptide nutritional group, the subjects put 10 g of the oligopeptide nutritional powder into a cup, poured 150 mL of plain boiled water, stirred to dissolve and drink.

Dinners started at 18:00 and ended at 20:00. Each of 30 subjects drank 200 mL of liquor (Kweichow Moutai Liquor 2018, 53% vol. 106 PROOF) or 400 mL of red wine (France Saint Arnaud Cabernet Sauvignon 2015, 13.5% vol. Cabernet Sauvignon) within two hours. The subjects' dinners were mainly Chinese stir-fried dishes, including fish, meat and vegetables, with rice as the staple food, and the dinners lasted two hours. Every 30 minutes after the start of drinking, the breath alcohol tester (Mr. Black-03) was used to test the blood alcohol content of each subject for four hours, then test once every two hours; on the 16^{th} hour after drinking, the test was over. The data of the subjects were recorded separately, and the statistical analysis of data was performed by groups. The blood alcohol metabolism curve of each group was completed by Gaussian Fitting method. For oligopeptide nutritional powder, after 4 hours of excessive drinking of liquor and red wine, namely, 2 hours after the end of meals, the blood alcohol content was measured, as shown in Table 3.

As shown in Table 3, within 4 hours of drinking, the blood alcohol content of the subjects who took 10 g of the oligopeptide nutritional powder was lower than the alcohol content threshold for drunk driving (20 mg/100 mL), while the control group and the cysteine group The blood alcohol content of the subjects was higher than the alcohol content threshold for drunk driving, namely, taking 10 g of the oligopeptide nutritional powder had a significant effect on sobering and hangover.

TABLE 3
Effect of oligopeptide nutritional powder on blood alcohol content
4 hours after excessive drinking of liquor and red wine

| | | | Oligopeptide |
|---|---|---|---|
| Blood alcohol | Control group | Cysteine | nutritional |
| content | (no health | group | powder |
| (mg/100 mL) | care products) | (1200 mg) | (10 g) |
| Blood alcohol content 4 | 53.6 ± 2.5^{a} | 42.3 ± 3.1^{b} | 8.6 ± 1.9^{c} |
| hours after | | | |
| drinking liquor | | | |
| Blood alcohol content 4 | 46.1 ± 3.2^{a} | 38.7 ± 6.8^{ab} | 11.7 ± 2.5^{c} |
| hours after | | | |
| drinking red wine | | | |

Significant difference comparison (P < 0.05)

It will be obvious to those skilled in the art that changes and modifications may be made, and therefore, the aim in the appended claims is to cover all such changes and modifications.

The invention claimed is:

1. A composition of matter, comprising, by weight:
oligopeptide powder 55-80%;
fruit powder 5-30%;
amino acid powder 0.5-5%;
multivitamin powder 1.25-5%;
mineral powder 0.25-3%; and
compound sweetener powder 5-10%.

2. The composition of claim 1, wherein the oligopeptide powder comprises the following components by weight: soybean oligopeptide powder 40-55%, shad egg oligopeptide powder 10-20%, corn oligopeptide powder 10-20%, ginseng oligopeptide powder 5-10%, rice oligopeptide powder 5-10%, and walnut oligopeptide powder 5-10%.

3. The composition of claim 1, wherein the fruit powder comprises the following components by weight: Hovenia dulcis powder 20-40%, sea buckthorn powder 20-30%, pear powder 20-30%, milk thistle powder 5-10%, Aronia melanocarpa powder 5-20%.

4. The composition of claim 1, wherein the amino acid powder comprises the following components by weight: cysteine powder 40-60%, proline powder 20-40%, and threonine powder 10-30%.

5. The composition of claim 1, wherein the multivitamin powder is a complex B vitamin and comprises the following components by weight: vitamin B1 20-40%, vitamin B2 20-40%, vitamin B3 10-20%, vitamin B6 15-25%, vitamin B9 1.5-5%, vitamin B12 0.5-2%, and vitamin B7 0.2-0.8%.

6. The composition of claim 1, wherein the mineral powder comprises the following components by weight: food-grade zinc gluconate powder 30-70%, and food-grade magnesium sulfate powder 30-70%.

7. The composition of claim 1, wherein the compound sweetener powder is Momordica grosvenori powder, arabinose, or a mixture thereof.

8. A method for hangover cure comprising administering to a drunken person in need thereof the composition of matter of claim 1.
